(19) United States
(12) Patent Application Publication
(10) Pub. No.: US 2024/0246647 A1
(43) Pub. Date: Jul. 25, 2024

(54) REBREATHER AND PROCESS FOR CHECKING THE FUNCTIONAL READINESS OF A REBREATHER

(71) Applicant: Dräger Safety AG & Co. KGaA, Lübeck (DE)

(72) Inventors: Helge TRABERT, Lübeck (DE); Michael BRODERSEN, Lübeck (DE); Marcel WAßMER, Lübeck (DE)

(21) Appl. No.: 18/415,772

(22) Filed: Jan. 18, 2024

(30) Foreign Application Priority Data
Jan. 25, 2023 (DE) 10 2023 101 813.2

(51) Int. Cl. B63C 11/14; B63C 11/02

(52) U.S. Cl. CPC B63C 11/14; B63C 2011/021

(57) ABSTRACT

A rebreather (1) has at least the following components: a control unit (10), a low pressure system (77) with a breathing bag (3), a low pressure sensor (41), a valve arrangement (7), a hose line system (17), a breathing circuit (15), a mouthpiece (19), a CO2 absorber (13), a high pressure system (79) with a compressed gas supply (5), a pressure reducer (34), and a high pressure sensor (43). The control unit (10) is configured to carry out a procedure for checking the low-pressure system (77).

### CROSS REFERENCE TO RELATED APPLICATIONS

This application claims the benefit of priority under 35 U.S.C. § 119 of German Application 10 2023 101 813.2, filed Jan. 25, 2023, the entire contents of which are incorporated herein by reference.

### TECHNICAL FIELD

The invention relates to a process for checking the functional readiness of a rebreather apparatus (rebreather) and a rebreather with a circuit for breathing gas.

Rebreathers are often also referred to as closed-circuit breathing apparatus, LDBA (LDBA=Long Duration Breathing Apparatus) or SCBA (SCBA=Self Contained Breathing Apparatus). According to the invention, the rebreather is configured to carry out a check of the functional readiness of the rebreather exclusively with means which are arranged as components in or on the rebreather for operating the rebreather in an application. The means which make the check possible comprise as essential components a control unit and a sensor system, in particular a pressure sensor system.

### BACKGROUND

The process according to the invention makes it possible to check the functional readiness of a closed-circuit breathing apparatus using only components of the closed-circuit breathing apparatus. In a closed-circuit breathing apparatus, in particular in rebreathers, in order to reduce the weight and increase the operating time, breathing takes place in the circuit and only the oxygen consumed in each case is replaced from a compressed gas supply and fed into the circuit. An accumulation of the exhaled carbon dioxide (CO₂) in the circuit to physiologically harmful levels must be avoided. For this purpose, the circuit contains a CO₂ absorber with an absorbent that removes the CO₂ from the breathing circuit. The quantities of oxygen consumed by the breathing and metabolism of the equipment wearer-and exhaled as carbon dioxide and removed from the exhaled gas by the CO₂ absorber-must be replaced by fresh oxygen. This is done by replenishing oxygen from the compressed gas supply. This replenishment takes the form of dosing oxygen into a breathing bag in the rebreather by means of a valve. The breathing bag is a flexibly configured bag into which the air exhaled by the user is absorbed by the CO₂ absorber after the CO₂ has been removed and is then returned to the circuit. Dosing into the breathing bag is usually carried out using a valve arrangement. The valve assembly doses oxygen into the breathing bag or into the breathing circuit in such a way that the breathing bag is always kept full. The amount of oxygen required by the user's respiration is replenished by the dosing. In order to avoid any possibility of harmful gases entering the breathing protection system circuit and thus reaching the user, the closed-circuit breathing apparatus is operated in such a way that there is an overpressure (positive pressure) in the circuit compared to the operating environment. This means that any loss of gas in the circuit leads to a replenishment of oxygen through dosing into the circuit. In order to take into account the varying degrees of respiration of the user and also the leaks and leakage, the dosing into the breathing bag or into the breathing circuit can be carried out according to two different principles of device operation, i.e. by means of constant dosing or by means of on-demand dosing. These two principles can be implemented in various ways in designs of rebreathers as independent operating modes or in combination with each other. For example, a first means of dosing can be used to dose continuously from the compressed gas supply into the breathing bag or into the breathing circuit at an essentially constant flow rate, for example in the range of 1 l/min to 2 l/min.

With a second means for dosing, an additional amount of oxygen can be dosed on demand when the breathing bag is almost empty. The breathing bag is provided with a lever construction for this purpose, which is connected to the second means for dosing, so that the second means for dosing is coupled to the movement of the breathing bag and allows a further additional quantity of oxygen to flow into the breathing bag when the breathing bag is almost empty. Such a second means of dosing is also referred to as a minimum valve and is usually opened by means of a lever construction arranged on the breathing bag as soon as the breathing bag has largely collapsed, i.e. only a small volume of breathing gas is still stored in the breathing bag. This means that in addition to the continuously dosed, essentially constant volume flow, a further quantity of oxygen can be dosed and supplied to the circuit. Generally known devices have a permanent pressure measurement and display (manometer), which constantly informs the user about the available breathing gas supply. A warning device indicates when the breathing gas supply is running low. Such pressure gauges as well as such warning devices can be mechanical/pneumatic or configured with electronic components and sensors.

U.S. Pat. No. 4,364,384A shows a pressurized oxygen circuit breathing apparatus with positive pressure in the breathing circuit, which has a valve, whereby this valve is configured with an additional functionality in the mechanical lever construction, so that when the breathing bag is completely empty, this valve closes, and the dosing is terminated. This ensures that the compressed gas supply is not completely emptied in the event of major leaks.

DE 82 01 997 U1 shows a breathing apparatus with a breathing air circuit. Air and oxygen from a compressed air cylinder and an oxygen cylinder are fed into the breathing bag by means of an automatically controlled equalizing gas supply valve and a switching valve.

DE 34 29 345 C2 shows a closed-circuit breathing apparatus which can be operated in overpressure mode, whereby movements of the breathing bag via a pressurized gas line by means of an auxiliary device lead to an increase in pressure in the breathing circuit. Furthermore, a measuring circuit is provided to recognize breathing phases, so that in the exhalation phase the auxiliary device causes the pressure increase at the breathing bag.

U.S. Pat. No. 10,252,089 B2 shows a monitoring device for monitoring a wearer of a respirator, wherein the monitoring device is arranged to determine whether breathing has started using the respirator and to activate the dosing of quantities of breathing gas if the breathing of the wearer has started.

U.S. Pat. No. 10,183,184 B2 shows a method for operating a closed-circuit breathing apparatus, wherein a pressure measurement within the breathing circuit using a pressure sensor is used to determine whether sufficient quantities of breathing gases are supplied into the breathing circuit, wherein a warning is issued on the basis of the pressure measurement if insufficient quantities of breathing gases are supplied into the breathing circuit.

Further prior art on rebreathers can be found in U.S. Pat. Nos. 4,879,996 A, 5,048,517 A and 4,266,539 A. Various systems are known for cooling the breathing air. One common process is cooling with ice, as described in DE 33 45 584 C1, for example. Alternative options for cooling breathing gases include zeolite-water coolers, evaporative coolers or wax coolers.

Reliable operational and functional readiness of rebreathers is of great importance for the wearer. The wearer must be certain that the rebreather intended for use is ready for use. In normal operations, the equipment is cleaned, checked and prepared for the next use by a so-called equipment maintenance person after use. The control unit and the internal sensors are used, for example, to check the tightness of the high-pressure system and/or low-pressure system, as well as to check the correct configuration of the device's internal components, such as pneumatic or electrical line connections, breathing bag, breathing gas cooler, CO₂ absorber, valves of the valve arrangement, compressed gas cylinder, control unit, display unit or control and display unit, energy storage unit and the sensors themselves in the housing shell. In addition, an external test (checking) device can be used to check whether breathing activity is detected by the device carrier using the sensors and control unit. The function of detecting breathing activity is essential when using the rebreather so that the wearer can be notified or alerted if they have started breathing in the circuit and the compressed gas cylinder is either not open or is already empty. Therefore, in addition to the aforementioned leakage tests, it is also very important that this function is checked by the equipment maintenance as part of the equipment preparation. The external test device can be used to simulate breathing on the rebreather.

### SUMMARY

The disadvantage of using such an external test device is that such an external test device is not available in every situation. The present invention has therefore addressed the issue of providing a way to check the function of a respiratory activity detection without an external test device. A secondary condition for a successful solution to this problem, which is also reflected in the way in which the solution according to the invention is configured, is the fact that a deterministic methodology for detecting respiration activity implemented in the control unit does not have to be checked per se, but it only has to be ensured that the measurement signals or data of the low-pressure sensor are available without errors for applying the methodology. Since pneumatic lines between the low pressure sensor and the measuring point of the pressure in the low pressure system as well as electrical lines between the low pressure sensor and electronics with signal acquisition and signal processing in the control unit must also be checked during cleaning and reprocessing of a rebreather, replaced if necessary and, depending on the condition, also re-routed or re-plugged, the task of the present invention is also extended in that the correct configuration and proper assembly after completion of maintenance activities on the rebreather before re-use is also reliably enabled without the need for an external test device. In connection with the testing of pneumatic lines, correct plugging of connectors/coupling elements must also be ensured, in particular if self-closing pneumatic connectors are used.

The object of the present invention is therefore to provide a process for checking (testing) the operational readiness of a rebreather.

A further object of the present invention is to provide a computer program or a computer program product which enables the functional readiness of a rebreather to be checked.

A further object of the present invention is to provide a rebreather whose functional readiness can be checked internally.

The task of providing a process for checking a rebreather is solved by a process comprising the steps of: continuously metrologically detecting and recording of pressure measurement signals of a low-pressure sensor arranged in or on a low-pressure system of the rebreather; identifying, based on the pressure measurement signals of the low pressure sensor as to whether there is currently a situation in which an inflow of quantities of gas into the low pressure system has started; storing a first pressure measurement signal at a first time, which indicates the identified pressure situation immediately after the start of the inflow of gas quantities into the low-pressure system with a breathing bag; storing a second pressure measurement signal at a second time, which indicates a pressure situation at the end of the inflow of gas quantities into the low-pressure system; forming a differential value between the first pressure signal and the second pressure signal; carrying out a comparison between the difference value and a difference threshold value; determining a result of checking the functionality of the rebreather based on the comparison; and providing an output signal which indicates the result of the check.

The tasks of providing a computer program or a computer program product for checking a rebreather are solved by a computer program with non-transitory computer-readable media or computer program product with non-transitory computer-readable media for performing one or more of the process steps.

The task for a closed-circuit breathing apparatus with internal functional readiness check is solved with a device for performing the processes and with a closed-circuit breathing apparatus with the device is configured as a closed-circuit breathing apparatus, rebreather apparatus or rebreather diving apparatus, which in one configuration has at least the following components: a control unit; a low-pressure system comprising a breathing bag, the low-pressure sensor, a valve arrangement, a hose line system, a breathing circuit, a mouthpiece, and a CO₂ absorber; and a high-pressure system with a compressed gas supply, a pressure reducer, and the high-pressure sensor.

The process according to the invention makes it possible to check the functional readiness of the rebreather without an external test device. The closed-circuit breathing apparatus according to the invention is configured to enable the functional readiness of the closed-circuit breathing apparatus to be checked without an external test device by means of a control unit and an internal sensor system configured for pressure measurement.

Advantageous embodiments of the invention are disclosed and are explained in more detail in the following description with partial reference to the figures. Features and details which are described in connection with the rebreather naturally also apply in connection with the process according to the invention for checking the functional readiness of the rebreather and vice versa, so that reference is or can always be made mutually to the individual aspects of the invention with regard to the disclosure.

A rebreather with a breathing circuit has the following essential components:
a hose line system (tubing system) with a mouthpiece for supplying quantities of breathing gases as inhaled gas to a device wearer and for transporting (guiding) quantities of exhaled breathing gases from the device wearer out of the breathing circuit,
a compressed gas cylinder filled with oxygen at a high pressure above 100 hPa as a compressed gas supply,
a pressure reducer to reduce the high pressure to a low pressure in the range slightly above the pressure of the environment in which the equipment carrier is used,
a CO₂ absorber, which serves to chemically bind CO₂ in the breathing gas and represents a heat source due to the reaction heat generated during the chemical binding of CO₂,
a breathing bag to buffer quantities of breathing gases in the breathing circuit,
an optional breathing gas cooler functioning as a heat sink, which is arranged in the direction of circulation in the breathing circuit behind the heat source and in front of the mouthpiece in order to cool heated quantities of breathing gases during operation,
a valve arrangement with at least one dosing valve for dosing quantities of oxygen from the compressed gas supply into the breathing circuit or into the breathing bag by means of the pressure reducer,
an internal sensor system, in particular a pressure sensor system and a control unit for monitoring during operation and for checking the functions of the rebreather,
at least one display unit to provide instructions and alarms to the device operator and to guide the device operator when carrying out test or inspection routines,
at least one control unit for operating the functions of the rebreather, for carrying out test or inspection routines and for acknowledging notifications and/or alarms,
at least one energy storage unit to supply the control unit, sensor system, control unit and display unit with electrical energy,
in addition, in conventional embodiments, a housing shell for accommodating the aforementioned components and
a carrying frame connected to the housing shell with a belt system for use of the rebreather by the wearer during use.

The control unit and display unit can also be configured as a combined control and display unit. To begin with, some of the terms used in this patent application and the basic principle of a closed-circuit breathing apparatus are explained in more detail. For the purposes of the present invention, a high pressure is to be understood as a pressure range from 100 hPa to 300 hPa. For the purposes of the present invention, a low pressure is to be understood as a pressure range with a difference of more than 1 hPa and less than 10 hPa above the pressure in the environment in which the device carrier is used.

A high-pressure system of the rebreather is formed by the compressed gas cylinder filled with oxygen under high pressure with a manual cylinder valve for opening the compressed gas cylinder, a pressure reducer which is configured to reduce the high pressure to a low pressure in a range of the ambient pressure, high-pressure line connections between the cylinder valve and the pressure reducer and a pressure sensor arranged on the high-pressure system—hereinafter referred to as high-pressure sensor—which is provided and suitably configured to detect a pressure level given in the high-pressure system. The high-pressure sensor is configured to provide signals or data indicating a pressure level in the high-pressure system by means of data transmission to the control unit.

A low-pressure system of the rebreather is formed by the hose line system with the mouthpiece for coupling to a breathing mask of the wearer, the breathing bag, the valve arrangement, the CO₂ absorber, the optional breathing gas cooler, low-pressure line connections between pressure reducer, breathing bag, CO₂ absorber, breathing gas cooler, valve arrangement and a pressure sensor arranged on the low-pressure system-hereinafter referred to as low-pressure sensor—which is intended and suitably configured to detect a pressure level in the low-pressure system, as well as the breathing mask itself if it is in contact with the mouthpiece. The low pressure sensor is configured to provide signals or data that indicate a pressure level in the low pressure system by means of data transmission to the control unit. The low pressure sensor can be located at various measuring points in the breathing circuit or on components of the low pressure system. Examples of suitable measuring locations are the breathing bag, the CO₂ absorber, the breathing gas cooler or the valve arrangement. The choice of measuring location for the low-pressure sensor in the breathing circuit is based on the criteria of maintainability, installation space and accessibility in the device as well as signal quality and the lowest possible susceptibility to errors and faults in the technical realization of closed-circuit breathing apparatus. From the point of view of good maintainability, coupling the low-pressure sensor to the breathing gas cooler is advantageous, in particular coupling the low-pressure sensor to the CO₂ absorber is also advantageous.

In optional variants of the closed-circuit breathing apparatus, a pre-purge device can also be provided which, when the closed-circuit breathing apparatus is started up—activated by opening the cylinder valve of the compressed gas cylinder—ensures an inflow of oxygen for a predetermined period of time so that the breathing bag is filled with a defined volume of oxygen before breathing begins in the breathing circuit. Such a pre-purge device is particularly important in variants of the closed-circuit breathing apparatus without constant dosing, so that the wearer of the apparatus is supplied with oxygen directly at the start of breathing in the breathing circuit, thus preventing the wearer from inhaling significant quantities of previously exhaled breathing gas again.

In special variants or configurations of the rebreather, an optional manual dosing element (bypass element) can be provided in the low-pressure system. Such a manual dosing element (bypass element)—usually configured as a control button for manual activation—enables the wearer to dose an additional amount of oxygen into the low-pressure system and thus directly into the breathing circuit when required. This can improve comfort for the wearer in situations with very high physical exertion. In addition, the manual dosing element (bypass element) can be used to vent the high-pressure system when the cylinder valve of the compressed gas cylinder is closed.

The control unit is configured to coordinate an operation and/or routines for checking functions of the rebreather. The control unit is configured to receive the data or signals from the high pressure sensor and the low pressure sensor, to prepare them by means of signal processing (A/D conversion) and/or signal amplification (OP amps) and/or signal filtering, to process them by means of a computing unit and to carry out evaluations with the processed data. The computing unit can, for example, be configured as a processor unit (μP, μC) with an associated data memory (RAM, ROM).

For example, the computing unit-and therefore also the control unit—can be set up and programmed using a program code to evaluate data or signals from the low pressure sensor to determine whether the wearer has started breathing.

The control unit or the computing unit can, for example, also be set up or programmed to evaluate data or signals from the high pressure sensor to determine whether the pressure in the high pressure system is sufficient for imminent use of the rebreather on the device carrier. For example, a value of 150 Pa can be used by the control unit as a lower threshold value for sufficient filling of the compressed gas supply. The valve arrangement comprises at least one dosing valve, preferably a dosing valve for constant dosing, a minimum valve that can be activated by movements of the breathing bag and a pressure relief valve for relieving the breathing bag, for example in the event of a fault in the compressed gas supply. To enable the breathing bag to be filled and emptied with a cyclical movement in the circuit of inhaled and exhaled air into and out of the breathing bag, the rebreather has a base plate, a spring bridge, a breathing bag plate and a spring arrangement as further components. The base plate and the spring bridge as a type of mounting bracket are arranged at a predetermined distance from each other in the rebreather. The breathing bag is held on the base plate on one side (underside), and the breathing bag plate is arranged on the breathing bag on the second side (upper side) opposite this one side. The spring arrangement is arranged between the breathing bag plate and the spring bridge with a predetermined spring tension, so that the support of the spring arrangement on the spring bridge exerts a force on the breathing bag plate and thus also on the breathing bag itself, so that a certain pressure is applied to the breathing bag by means of the predetermined spring tension. This pressure on the breathing bag causes an overpressure in the breathing bag in a pressure range of 1 hPa to 10 hPa compared to the environment. During use, the rebreather is used by a user in such a way that the required amount of breathing gas is taken from the breathing bag of the rebreather via the hose line system and the mouthpiece as a connecting element to a mask via the inhalation valve with the inhalation, with flow through an optional breathing gas cooler, and returned to the breathing bag of the rebreather via the exhalation valve with the exhalation. Gas flows into the breathing bag and out of the breathing bag through the user's breathing.

As a result, a quantity of air in the breathing bag is cyclically moved back and forth in the circuit, whereby carbon dioxide is removed from the circuit by the CO₂ absorber and oxygen is added in sufficient quantity from the compressed gas supply by means of at least one metering valve, thus compensating for the eliminated carbon dioxide so that fresh breathing gas is available in the breathing bag. The positive pressure in the breathing bag ensures that this positive pressure is present in the user's mask and that leaks in the mask cannot lead to ambient air flowing into the mask. This is particularly important as the ambient air may contain harmful substances that could pose a health risk to the user if inhaled.

In a process according to the invention for checking the operational readiness of a rebreather, the following steps are carried out in a sequence of steps 1-8:
1. Continuous, metrological acquisition and recording of pressure measurement signals P_{L} of a low pressure sensor arranged in or on a low pressure system of the rebreather,
2. Identification, based on the pressure measurement signal P_{L} of the low pressure sensor, whether there is currently a situation in which quantities of gas are beginning to flow into the low-pressure system, which has a breathing bag,
3. Storage of a first pressure measurement signal P₁ at a first time t₁, which indicates the identified pressure situation immediately after the start of the inflow of gas quantities into the low-pressure system,
4. Storage of a second pressure measurement signal P₂, which indicates a pressure situation at the end of the inflow of gas quantities into the low-pressure system at a second point in time t₂,
5. Formation of a differential value ΔP (ΔP=P₂−P₁) between the first pressure signal P₁ and the second pressure signal P₂,
6. Carrying out a comparison between the difference value ΔP and a difference threshold value P_{C},
7. Determining a result of the functional capability check of the rebreather based on the comparison,
8. Provision of an output signal that indicates the result of the check.

The above-mentioned sequence of steps of the process can, for example, be carried out by the control unit—previously explained in the context of the description of the present invention for checking the operational readiness of a rebreather. A rebreather can be configured with such a control unit, which is configured and intended to coordinate and/or monitor operation of the rebreather during use and also to carry out a check of the rebreather.

To enable changes in the values of the continuously recorded measured values P_{L} of the low pressure sensor, a situation must exist in the rebreather in which quantities of breathing gas are currently flowing into the low pressure system, in particular into the breathing bag. Such a situation can arise, for example, when the cylinder valve of the compressed gas supply of the rebreather is opened. When performing the comparison between the differential value ΔP and the differential threshold value P_{C}, the control unit determines whether there is a difference ΔP in the pressure level of the low-pressure system between the start of the inflow and the end of the inflow into the breathing bag.

For example, a predetermined value in a range from 0.75 hPa to 1.5 hPa, preferably a value of 1.0 hPa, can be selected as the differential threshold value P_{C}.

If—despite the breathing bag being filled—no difference ΔP in the pressure level of the low-pressure system can be determined by the control unit, a situation may exist in which the low-pressure sensor was unable to correctly measure the pressure level in the low-pressure system, at least during the performance of the check at the first and/or second time t_{L}, t₂. Such a situation may for example, indicate a defect in the low pressure sensor or be an indication that the low pressure sensor is not correctly coupled fluidically or pneumatically to the low pressure system, for example to the breathing bag or the CO₂ absorber, which may be caused, for example, by a pressure measurement line not being correctly connected to the CO₂ absorber, for example by means of a pneumatic plug connection, and the low pressure sensor therefore not being able to measure pressure values from the low pressure sensor that indicate a pressure level in the low pressure system.

If the pressure difference ΔP exceeds the differential threshold value P_{C}, the low pressure sensor is functioning correctly. In such a case, a positive check (test) result (pass) can be provided, for example on a control and display unit.

If the pressure difference ΔP does not exceed the differential threshold value P_{C}, a negative check (test) result (fail) is provided, e.g. as an output on the control and display unit.

In a preferred embodiment, a first low pressure comparison of the measured values P_{L} of the low pressure sensor with a first lower low pressure threshold value P_{A} can be carried out at the first time t₁—for example by means of execution by the control unit. For example, a predetermined value in a range from 1.8 hPa to 2.2 hPa, preferably a value of 2.0 hPa, can be selected as the first lower threshold value P_{A}.

If the measured value P_{L} of the low pressure sensor exceeds the first lower low pressure threshold value P_{A} at the first time t₁, a situation exists in the rebreather in which quantities of breathing gas are currently flowing into the low pressure system, in particular into the breathing bag. This means that further testing of the rebreather can be continued. In such a case, a positive check result (pass) can also be provided, for example on a control and display unit.

If the measured value P_{L} of the low pressure sensor does not exceed the lower low pressure threshold value P_{A}, there is no situation in the rebreather in which quantities of breathing gas can currently flow into the low pressure system, in particular into the breathing bag. In such a case, a negative check result (fail) can be provided on a control and display unit, for example. After troubleshooting the rebreather by maintenance personnel, the functionality of the rebreather is usually checked again.

In a further preferred embodiment of the process, a second low pressure comparison of the measured values P_{L} of the low pressure sensor with a second lower low pressure threshold value P_{B} can be carried out at the second time t₂—for example by means of execution by the control unit. For example, a predetermined value above the pressure level of the first lower threshold value P_{A}, preferably a value of 2.5 hPa, can be selected as the second lower threshold value P_{B}.

If the measured value P_{L} of the low pressure sensor exceeds the second lower low pressure threshold value P_{B} at the second time t₂, the breathing bag is sufficiently filled with breathing gases. The second low pressure value P₂ indicates a pressure level in the low pressure system at the end of the filling of the low pressure system with a sufficient amount of respiratory gas for the start of respiration. This allows further testing of the rebreather to be continued and a positive check result (pass) to be provided, for example on a control and display unit.

If the measured value P_{L} of the low pressure sensor does not exceed the second lower low pressure threshold value P_{B}, the breathing bag is not sufficiently filled for respiration to begin. In such a case, a negative check result (fail) can be provided, for example on a control and display unit. After troubleshooting of the rebreather by maintenance personnel, the functionality of the rebreather is usually checked again.

In a further preferred embodiment of the process, a relief valve and/or drainage valve arranged at an outlet of the breathing bag can be checked at a third time t₃—for example by means of implementation by the control unit—a third low pressure comparison of the measured values P_{L} of the low pressure sensor with an upper low pressure threshold value P_{E}. On the one hand, a relief pressure valve arranged in or on the breathing bag is checked, which in typical embodiments allows excess pressure of breathing gas to escape from the breathing bag at a pressure level P_{L} in the breathing bag of above 8 hPa. On the other hand, a drainage valve arranged in or on the low-pressure system for draining accumulated amounts of liquid in the breathing bag is checked, which in typical embodiments allows excess liquid to flow out of the breathing bag at a pressure level P_{L} in the breathing bag in a range above 15 hPa to 20 hPa. For example, a predetermined value in a range from 7 hPa to 20 hPa, preferably a value of 16 hPa, can be selected as the upper threshold value P_{E}.

If the measured value P_{L} of the low pressure sensor does not exceed the upper low pressure threshold value P_{E} at the third time t₃, the relief valve on the breathing bag and/or the drainage valve are functioning correctly. In such a case, a positive check result (pass) can be provided on a control and display unit, for example.

If the measured value P_{L} of the low pressure sensor exceeds the upper low pressure threshold value P_{E} at the third time t₃, the relief valve on the breathing bag and/or the drainage valve are functioning correctly. In such a case, a negative check result (fail) can be provided on a control and display unit, for example.

In a further preferred embodiment of the process, the process for checking the functional readiness of the rebreather can also include a check of a manual dosing element (bypass element) arranged in the low-pressure system. For example, the control unit can perform a check by comparing the current low pressure level in the low pressure system P_{L}. If an increase in the pressure level in the low-pressure system by a predetermined pressure difference P_{E} is identified when the manual dosing element is activated, the check of the manual dosing element (bypass element) has been successfully completed and a positive check result (pass) can be provided, for example on a control and display unit.

If no increase in the pressure level in the low-pressure system by the predetermined pressure difference P_{E} is identified when the manual dosing element is activated, there is a negative check result in relation to the manual dosing element (bypass element). A negative check result (fail) can be provided on a control and display unit, for example.

In a further preferred embodiment, the process for checking the functional readiness of the rebreather can also include a check of the high-pressure system. In a first variant of checking the high-pressure system, a filling pressure of the compressed gas supply can be checked when the cylinder valve of the compressed gas supply is open. For this purpose, a high-pressure sensor is arranged on the compressed gas supply or in the high-pressure system. For example, the control unit can perform a check by comparing the currently detected high-pressure level in the high-pressure system P_{H} with a lower high-pressure threshold value. Such a lower high-pressure threshold value can be defined, for example, as a value P_{D} in a pressure range of 150 Pa to 180 Pa.

If the pressure level of the high-pressure system is above the upper high-pressure threshold value P_{D}, the check has been successfully completed, i.e. there is a sufficient pressure level in the compressed gas supply for use of the rebreather, and a positive check result (pass) can be provided, in an optional embodiment with indication of the current high pressure P_{H} in the compressed gas supply, for example on a control and display unit.

If the pressure level of the high-pressure system is below the high-pressure threshold value P_{D}, there is a negative check result with regard to the filling of the compressed gas supply, and a negative check result (fail) can be provided, for example on a control and display unit.

In a second variant of checking the high-pressure system, the tightness of the high-pressure system can be checked. For example, pressure changes in the high-pressure system can be monitored by the control unit using an upper high-pressure change threshold value ΔP_{D}. In such a tightness check, in typical embodiments, after flooding of the high-pressure system with quantities of breathing gas under a high pressure P_{H}—caused by opening the cylinder valve on the oxygen pressure gas cylinder—and the resulting transfer of quantities of breathing gas under a low pressure P_{L} into the low-pressure system by means of the pressure reducer with the cylinder valve of the oxygen pressure gas cylinder closed again, pressure changes are monitored over a period of time t_{w}. A check is carried out over the period t_{w} to ensure that there is no deviation from the current measured values of the high pressure sensor P_{H} above the upper high pressure change threshold value ΔP_{D}.

Such an upper high-pressure change threshold value ΔP_{D} can, for example, be defined as a value P_{D} in a pressure range of 0.5 Pa to 1.5 Pa, preferably 1.0 Pa.

If there are no changes in the pressure level of the high-pressure system above the upper high-pressure change threshold value ΔP_{D} over the period t_{w}, the check has been successfully completed, i.e. no significant leaks have been identified in the high-pressure system during the check. This means that further testing of the rebreather can be continued and a positive check result (pass) can be provided on a control and display unit, for example.

If there are significant changes in the pressure level of the high-pressure system above the high-pressure change threshold value ΔP_{D} over the time period t_{w}, there is a negative check result with regard to the tightness of the high-pressure system and a negative check result (fail) can be provided, for example on a control and display unit.

In a particular embodiment, the high-pressure system can be checked as a precondition before the second variant is carried out in accordance with the first variant of checking the high-pressure system with regard to the sufficient filling pressure of the compressed gas supply, whereby the current high-pressure level P_{H} in the high-pressure system is compared with the lower high-pressure threshold value in a pressure range of 140 Pa to 180 Pa, preferably 150 Pa. This can, for example, be configured in such a way that the check of the tightness of the high-pressure system is only initiated by the control unit if a sufficiently high filling pressure of at least 150 Pa is present in the compressed gas supply (compressed oxygen gas cylinder) by means of the first variant of the check of the high-pressure system.

Based on the previously described embodiments of the checks of the low pressure system and the high pressure system, further preferred embodiments of checks of the low pressure system based on measured values P₁, P₂ of the low pressure sensor in comparison to associated pressure threshold values P_{A}, P_{B} or to the differential threshold value P_{C} can be carried out in a combination or in a common sequence with checks of the high pressure system based on measured values P_{H} of the high-pressure sensor in comparisons with high-pressure threshold values to associated pressure threshold values P_{D} or to the differential threshold value ΔP_{D}. In this way, sequences of checks with high-pressure and low-pressure systems can be formed in which the sequence of steps for checking the low-pressure system is combined or interleaved in a common sequence with steps for checking the high-pressure system, as well as in which the sequence of steps for checking the high-pressure system is combined or interleaved in a common sequence with steps for checking the low-pressure system.

A rebreather is configured with a control unit. According to the invention, the control unit is configured, constructed and intended to carry out the variants and embodiments described in connection with the process according to the invention for checking the functional readiness of a rebreather. The rebreather can be configured as a rebreather respirator or as a rebreather diving device. The closed-circuit breathing apparatus, the rebreather respirator or the rebreather diving device have at least the following components in one configuration:
a control unit,
a low-pressure system with breathing bag, low-pressure sensor, valve assembly, hose line system, breathing circuit, mouthpiece, CO₂ absorber,
a high-pressure system with compressed gas supply, pressure reducer, high-pressure sensor.

Optionally, the rebreather can also have a breathing gas cooler, a relief valve, a drainage valve and a manual dosing element (bypass element).

In a preferred embodiment, the rebreather can have an output unit which is arranged in or on the rebreather or in or on the control unit, which is in a data connection with the control unit and is configured to provide measurement signals, status data of the rebreather and/or results of checks of the functionality of the rebreather. For this purpose, in a preferred embodiment of an operating and output unit, the output unit can have a user interface, for example configured as a display unit for outputting acoustic alarm tones and/or for visual representations of instructions and/or alarms in the form of characters, digits, texts or symbols displayed in one or more colors for the device wearer or user. Alternatively or additionally, the output unit can have an interface, for example in the form of a wired or wireless interface for providing the notifications and/or alarms to an external evaluation unit, user, operations management or device control room. The operating and output unit can also be configured to instruct and guide the user, be it the device wearer and/or device maintenance attendant, through the routines for checking the functionality of the rebreather, for example to perform actions in certain situations in the procedures described above in various embodiments for checking the functionality of the rebreather, such as opening or closing the cylinder valve of the high-pressure gas cylinder or attaching or removing a stopper to the mouthpiece, as well as performing operating steps on the operating and output unit.

A significant advantage of the invention results from the fact that no additional external testing device is required to check the functional readiness of the detection of breathing activities of a device user in the low-pressure system.

Checking the low-pressure sensor with means that are permanently located in the rebreather itself allows a high degree of flexibility with regard to the time and location of the functional readiness check, be it during routine device maintenance, after use or on site before use.

In the following, exemplary embodiments of the invention are explained in more detail with reference to the figures, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic diagram of the basic structure of a rebreather;

FIG. 2 is a schematic diagram of a basic procedure for checking the functional readiness of a rebreather; and

FIG. 3 is a schematic diagram showing a variant of the process shown in FIG. 2.

### DESCRIPTION OF PREFERRED EMBODIMENTS

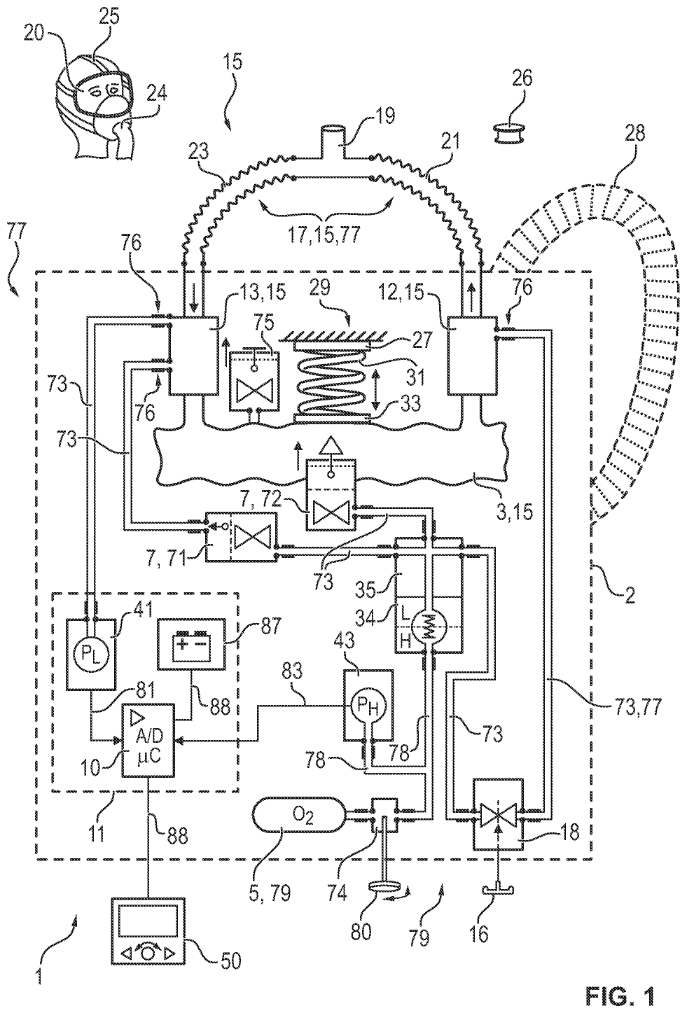

Referring to the drawings, FIG. 1 shows a rebreather 1 with a breathing circuit 15, with a control unit 10 for determining gas quantities, with a breathing bag 3, with a compressed gas supply 5, a valve arrangement 7, a CO₂ absorber 13 and an optional breathing gas cooler 12. The rebreather 1 can be connected via a hose line system 17 with an inspiratory branch 21 and an expiratory branch 23 and a mouthpiece 19 to a connection element 24 of a breathing mask 25 of a wearer of the device. The breathing mask 25 is shown here only schematically with connection element 24, a visor 20 and a strap 22. An electronic unit 11 with a control unit 10 and a low-pressure sensor 41 is indicated by a dashed line as an element of the rebreather 1. A high pressure sensor 43 is also assigned to the electronics 11. The electronics 11 also has means for data acquisition (A/D converter), signal amplification and signal filtering. The control unit 10 has a computing unit, for example in the form of a microcontroller (μC) or microprocessor (μP) and an associated data memory (RAM, ROM). A program code can be stored in the data memory (non-transitory and computer readable memory), which can be executed by the control unit 10 in conjunction with electronics 11 and sensors 41, 43 for operation and/or routines for checking functions of the rebreather 1.

The electronics 11 also has an energy storage unit 87 (battery). In addition, a control and display unit 50 is coupled to the electronics 11 or control unit 10. In the embodiment according to this FIG. 1, elements for operation and display are combined as a control and display unit; embodiments with a separately configured control unit and display unit are also possible within the meaning of the present invention. On the one hand, the control and display unit 50 has means which are suitable for outputting notifications and alarms. These include means for visual or acoustic output, for example for outputting visual representations of notices and/or alarms in the form of single-color or multi-color characters, digits, texts or symbols as suitable display means, as well as acoustic output means, such as horns, loudspeakers or piezo sound elements. On the other hand, the control and display unit 50 also has means for input and operation, such as touch or switching elements. The control and display unit 50 can also be configured as a graphical user interface (UI, GUI). In embodiments according to this FIG. 1, the energy storage unit 87 and the low pressure sensor 41 are shown as part of the electronics 11 by way of example, the high pressure sensor 43 is shown in an embodiment outside the electronics 11, in the sense of the present invention embodiments are also included in which at least one of the two pressure sensors 41, 43 is arranged inside the electronics 11, as well as embodiments in which at least one of the two pressure sensors 41, 43 is arranged outside the electronics 11. In addition, within the meaning (context) of the present invention, the control unit 10 and/or the power supply 87 can be arranged as a whole, predominantly or partially both as components of the electronics 11 and as components of the control and display unit 50. The control and display unit 50 is intended to enable the device wearer to carry out certain operating steps or settings on the rebreather 1, for example for configurations as well as acknowledgements of notifications or alarms, which are generated by the control unit 10 and provided to the control and display unit 50. The rebreather 1 has a construction-shown in this schematic representation according to FIG. 1—comprised of a base plate 27 and a spring bridge 29, via which the breathing bag 3 is arranged in the rebreather 1 by means of a spring arrangement 31 and a breathing bag plate 33 in such a way that the gas in the breathing bag 3 is kept at a pressure level above the ambient pressure. The wearer inhales fresh breathing gas via the inspiratory branch 21 of the hose line system 17 from the breathing bag 3 and through the optional breathing gas cooler 12 and via the expiratory branch 23 of the hose line system 17 and via the CO₂ absorber 13 back into the breathing bag 3 and thus into the breathing circuit 15. The valve arrangement 7 has a pressure reducer 34 and a first metering valve 71 as a means of adjusting the pressure from the compressed gas supply 5 to a pressure level that can be breathed by the user 25 for continuous constant metering into the low-pressure system 77, usually in the form of a pulsed metering valve 71, and a second metering valve 71 as a means of adjusting the pressure from the compressed gas supply 5 to a pressure level that can be breathed by the user 25, a second metering valve 72, which can be activated by coupling to deflections of the breathing bag 3 (not shown in this FIG. 1 for reasons of clarity), usually referred to as a minimum valve, for demand-controlled metering, i.e. metering dependent on the depth of respiration, i.e.

depending on the depth of respiration by the wearer of the device, to feed quantities of breathing gases into the breathing bag 3. A pressure relief valve 75 enables pressure to be released to the environment in the event of excess pressure in the breathing bag 3, which may be possible, for example, in the event of malfunctions at the pressure reducer 34. An optional manual dosing element (bypass element) 18 allows the wearer of the device to directly activate the dosing of gas quantities into the breathing circuit 15, shown in FIG. 1 with an exemplary feed at the breathing gas cooler 12. Other locations for feeding into the breathing circuit 15 or into a low-pressure system 77, for example into the breathing bag 3, are also technically possible. Pneumatic low-pressure lines 73 and a connection block 35 on the pressure reducer 34 pneumatically connect the components 3, 7, 12, 13, 18, 71, 72, 75, 76, 41 to each other and form the low-pressure system 77 with the components. The compressed gas supply 5, configured as a compressed oxygen gas cylinder with a filling pressure of above 200 hPa, forms a high-pressure system 79 with a cylinder valve 74 that can be operated by means of a handwheel 80, high-pressure lines 78 and the pressure reducer 34, the high-pressure sensor 43 and pneumatic high-pressure lines 78. The control unit 10 is connected to the low-pressure sensor 41 and the high-pressure sensor 43 by means of electrical signal lines 81, 83. The control unit 10 is electrically connected to other components of the electronics 11, such as the energy storage unit (battery) 87 and/or the control and display unit 50, by means of electrical connection lines 88. In this FIG. 1, a housing shell 2, in which the components described above are arranged, and a carrying system 28 for transporting the rebreather 1 on the back of a device carrier are shown in use in dashed lines. To check the functionality of the rebreather 1, the mouthpiece 19 of the hose line system 17 can be sealed using a sealing plug 26. In this way, the rebreather 1 can be tested-for example after maintenance—without pneumatic coupling to the device carrier. For example, leakage tests can be carried out in the high-pressure system 79 or in the low-pressure system 77 with the breathing circuit 15 sealed at the mouthpiece 19.

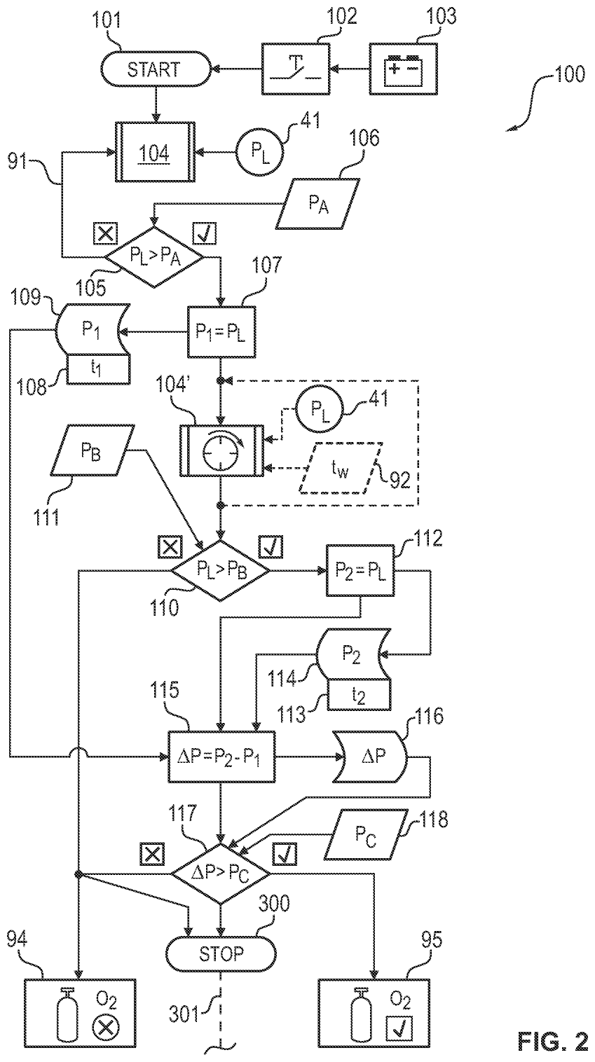

FIG. 2 shows in schematic form a basic sequence 100 of a procedure for checking the functional readiness of a rebreather 1 as shown in FIG. 1.

In order to be able to carry out this functional readiness check with the following sequence 100, the following boundary conditions are required:
The mouthpiece 19 (FIG. 1) on the hose line system 17 (FIG. 1) must be sealed by a sealing plug 26 (FIG. 1).
The low-pressure system 77 (FIG. 1) must be vented; the pressure in the low-pressure system 77 (FIG. 1) therefore corresponds to the ambient pressure.

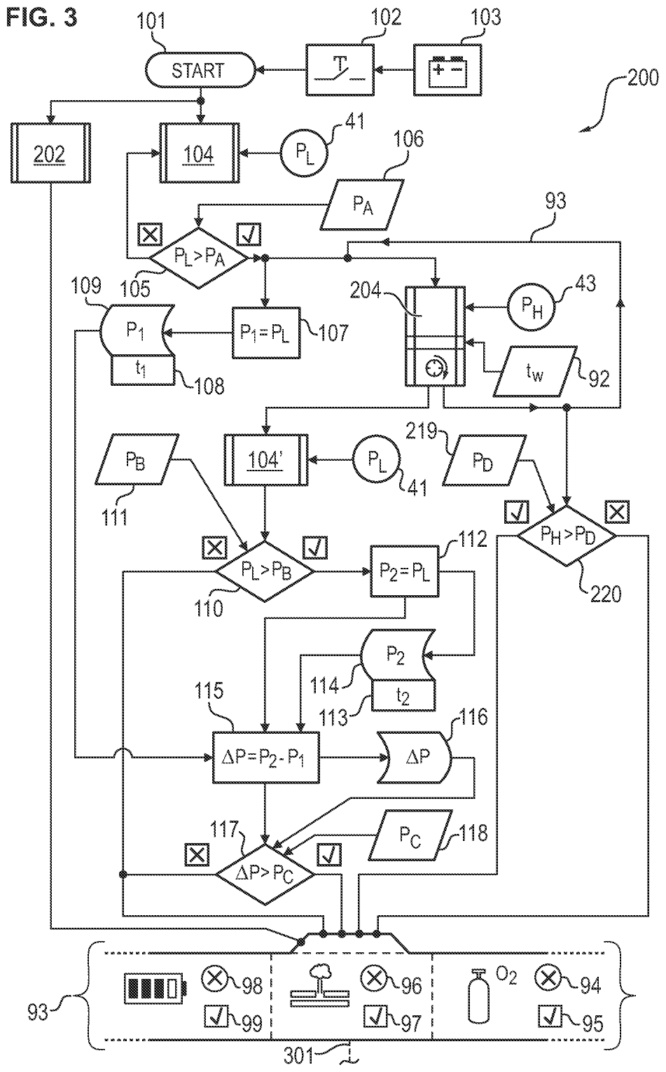

The sequence 100 begins with a start 101, the start of the rebreather 1 (FIG. 1) is initiated by a connection 102 of electrical energy 103. With the connection 102 of electrical energy 103, a routine starts in a continuous loop 91 with a continuous data acquisition 104 of measured values P_{L} of the low pressure sensor 41. At the same time as the data acquisition 104, a low pressure comparison 105 of the measured values P_{L} 41 with a first lower low pressure threshold value P_{A} 106 takes place continuously. If the measured value P_{L} of the low pressure sensor 41 exceeds the lower low pressure threshold value P_{A} 106, data storage 107 of the measured value P_{L} 43 as a first low pressure value P₁ 109 takes place at a first time t₁ 108. In order for the measured value P_{L} of the low pressure sensor 41 to reach the first lower low pressure threshold value P_{A} 106, a situation must exist in the rebreather 1 (FIG. 1) in which quantities of breathing gas are currently flowing into the low pressure system 77 (FIG. 1), in particular into the breathing bag 3 (FIG. 1). Such a situation can arise, for example, when the cylinder valve 74 (FIG. 1) is opened. The first low pressure value P₁ 109 indicates a pressure level in the low pressure system 77 (FIG. 1) at the start of the rebreather 1 (FIG. 1). The sequence 100 is continued immediately after the low pressure comparison 105 or also during the low pressure comparison 105 with continuous data acquisition 104′ of measured values P_{L} of the low pressure sensor 41. In an optional embodiment of the sequence 100, the continuous data acquisition 104′ can be performed limited to a predetermined time duration t_{w} 92 in a time loop 93. A second low pressure comparison 110 is then carried out during the continuous data acquisition 104′ of the measured value P_{L} of the low pressure sensor 41 with a second lower low pressure threshold value P_{B} 111. If the measured value P_{L} of the low pressure sensor 41 exceeds the second lower low pressure threshold value P_{B} 111, further data storage 112 of the measured value P_{L} 43 takes place at a second time t₂ 113 as a second low pressure value P₂ 114. The second low pressure value P₂ 114 indicates a pressure level in the low pressure system 77 (FIG. 1) at the end of the filling of the low pressure system 77 (FIG. 1), in particular of the breathing bag 3 (FIG. 1) with a quantity of breathing gas. If the measured value P_{L} of the low pressure sensor 41 does not exceed the second lower low pressure threshold value P_{B} 111, a negative check result 94 (fail) is output on a control and display unit 50 (FIG. 1), and the process 100 is terminated 300 (STOP). If the measured value P_{L} of the low pressure sensor 41 exceeds the second lower low pressure threshold value P_{B} 111, a difference 115 is formed on the basis of the first low pressure value P₁ 109 and the second low pressure value P₂ 114 with storage as pressure difference ΔP 116. A differential comparison 117 of the pressure difference ΔP 116 with a differential threshold value P_{C} 118 is then carried out. If the pressure difference ΔP 116 does not exceed the differential threshold value P_{C} 118, a negative check result 94 (fail) is output on the control and display unit 50 (FIG. 1). If the pressure difference ΔP 116 exceeds the differential threshold value P_{C} 118, a positive check result 95 (Pass) is output on the control and display unit 50 (FIG. 1). This ends the sequence 100 for checking the functional readiness of a rebreather 1 (FIG. 1) 300 (STOP) and, if the check result 95 is positive, a transition 301 to regular device operation or device provision can take place, or, if the check result 94 is negative, troubleshooting and possibly further checks on the rebreather 1 (FIG. 1) are usually carried out by maintenance personnel until all required check routines with positive results 95, 99 (FIG. 3), 97 (FIG. 3) have been passed.

FIG. 3 shows in schematic form a variant 200 of the sequence 100 according to FIG. 2 for an extended check of the functional readiness of a rebreather 1 according to FIG. 1. In addition to a check of the low pressure sensor 41, the sequence 200 also includes a check of the high pressure sensor 43. The sequence 200 and the start of the rebreather 1 (FIG. 1) starts in the same way as described for FIG. 2 with the start 201 of the rebreather 1 (FIG. 1) by connecting 102 electrical energy 103. The boundary conditions mentioned for FIG. 2 must also be given in the same way as described for FIG. 2 in order to be able to carry out the alternative sequence 200 for an extended check of the functional readiness. After the first data acquisition 104 of the low pressure sensor P_{L} 41 and the subsequent first low pressure comparison 105 with the first low pressure threshold value 106 as described for FIG. 2, a check of the high pressure system 79 (FIG. 1) is performed in addition to the check of the low pressure system 77 (FIG. 1). For this purpose, a continuous data acquisition 204 of measured values P_{H} of the high pressure sensor 43 (FIG. 1) takes place in a time loop 93 for a predetermined period of time 92. After the predetermined period of time 92 has elapsed, a check of the low pressure system 77 (FIG. 1) takes place as in the sequence 100 with the action elements 107, 104′, 112, 115, 300, comparison elements 110, 117 with threshold values 111, 118, data storage 108, 109, 113, 114, 116 and an output of the check results 94, 95. In addition, a high pressure comparison 220 of a measured value P of the high pressure sensor 43 recorded at the end of the predetermined time period 92 with a high pressure threshold value 219 is performed. If the measured value P_{H} of the high pressure sensor 43 does not exceed the lower high pressure threshold value P_{D}, a negative check result 96 (fail) is output on a control and display unit 50 (FIG. 1). If the measured value P_{H} of the high pressure sensor 43 exceeds the lower high pressure threshold value P_{D}, a positive check result 97 (Pass) is output on a control and display unit 50 (FIG. 1). In this FIG. 3, a type of provision 93 of the check results 93, 94, 95, 96, 97, 98, 99 is shown as an example, schematically and symbolically, as it can be realized on a control and display unit 50 (FIG. 1) with graphic output options. In this FIG. 3, a check of the energy supply, for example of a battery charge state directly after the start 201 of the sequence 200, is shown as an example of further checks; in this exemplary embodiment, the check results 98, 99 are then fed to the provision 93, for example as the situation of a battery storage unit 87 (FIG. 1) with a good charge state 99 or a charge state 98 that is too low. The steps following the provision 93 in this FIG. 3 can also be configured, for example, as described for FIG. 2 with a termination 300 (FIG. 2) of the process 100 (FIG. 1) or the process 200. In this FIG. 3—for reasons of clarity of the drawing of FIG. 3—only a transition 301 is indicated, which can be configured as a transition 301 to a regular device operation, to a device provision or as a transition to further tests. Interactions with a user, for example details of the electrical start-up 102, 103 of the rebreather 1 (FIG. 1) or opening or closing of the cylinder valve 74 (FIG. 1) by means of a handwheel 80 (FIG. 1) in the course of the sequence 200 of checking the low-pressure system 79 (FIG. 1), any actuations or acknowledgements of the user on the display and control unit 50 (UI, GUI) (FIG. 1) required in the sequence 200 are not shown in FIG. 3 for reasons of clarity of the drawings.

While specific embodiments of the invention have been shown and described in detail to illustrate the application of the principles of the invention, it will be understood that the invention may be embodied otherwise without departing from such principles.

### REFERENCE NUMBER LIST

1 Circulatory breathing apparatus, rebreather apparatus, rebreather diver
2 Housing shell (case)
3 Breathing bag, counterlung
5 Compressed gas supply, compressed oxygen gas cylinder
7 Valve arrangement
10 Control unit
11 Electronics, electronics module
12 Breathing gas cooler
13 CO₂—absorber, breathing soda lime container
15 Breathing circuit (breathing circuit in the low pressure system)
16 Control element (button) for bypass element
17 Hose line system (inhalation and exhalation hose)
18 Manual dosing element, bypass element (O₂—additional dosing)
19 Mouthpiece on the hose line system
20 Visor of the breathing mask
21 Inspiratory branch (inhalation tube)
22 Strapping of the breathing mask
23 Expiratory branch (exhalation tube)
24 Connection element on the breathing mask
25 Respirator mask, user
26 Sealing plug for mouthpiece on the hose line system
27 Base plate
28 Carrying system (straps, belt system)
29 Spring bridge arrangement (base plate, spring arrangement)
31 Spring arrangement, spring assembly
33 Breathing bag plate
34 Pressure reducer (device for reducing pressure)
35 Connection block/manifold (low-pressure distribution)
41 Low pressure sensor P_{L}
43 High pressure sensor P_{H}
50 Display and control unit (UI, GUI)
71 First dosing valve (constant dosing)
72 Second dosing valve, minimum valve (on-demand dosing)
73 Low-pressure lines/tubes/pipes in the low-pressure system
74 Gas-cylinder valve (shut-off valve)
75 Pressure relief valve (relief valve)
76 Pneumatic connecting elements (plug/coupling)
77 Low pressure system
78 High-pressure lines/tubes/pipes in the high-pressure system
79 High pressure system
80 Handwheel (rotary wheel)
81 Electrical signal line (low pressure sensor)
83 Electrical signal line (high pressure sensor)
87 Energy storage (battery)
88 Electrical connecting cables
91 Continuous loop
92 Predetermined time period tw
93 Time loop
93 Provision of test results, output or presentation
94 Negative check result Low pressure check
95 Positive check result Low pressure check
96 Negative check result High pressure check
97 Positive check result High pressure check
98 Negative result energy supply check (charge level low)
99 Positive result energy supply check (state of charge good)
100 Sequence (FIG. 2, FIG. 3) of the check (test)
101 Start (start of the low pressure check)
102 Connection (switch-on), switch actuation
103 Electrical energy, energy storage (battery)
104, 104′ Data acquisition P_{L}, low pressure
105 First low-pressure comparison, comparison
106 First lower low-pressure threshold P_{A}
107 Data storage Low pressure P₁
108 First time t₁
109 First low pressure value P₁
110 Second low-pressure comparison, comparison
111 Second lower low-pressure threshold P_{B}
112 Data storage low pressure P₂
113 Second time t₂
114 First low pressure value P₁
115 Differential formation low pressure
116 Pressure difference low pressure ΔP
117 Differential comparison low pressure
118 Differential threshold value low pressure P_{C}
200 Alternative test procedure (FIG. 3)
201 Start (start of the combined low-pressure/high-pressure check)
202 Checking the power supply (battery charge status)
204 Data acquisition PH, high pressure
219 Lower high pressure threshold P_{D}
220 High pressure comparison, comparison
300 STOP (end of check)
301 Transition

What is claimed is:

1. A process for checking the operational readiness of a rebreather, the process comprising the steps of:
continuously metrologically detecting and recording of pressure measurement signals of a low-pressure sensor arranged in or on a low-pressure system of the rebreather;
identifying, based on the pressure measurement signals of the low pressure sensor as to whether there is currently a situation in which an inflow of quantities of gas into the low pressure system has started;
storing a first pressure measurement signal at a first time, which indicates the identified pressure situation immediately after the start of the inflow of gas quantities into the low-pressure system with a breathing bag;
storing a second pressure measurement signal at a second time, which indicates a pressure situation at the end of the inflow of gas quantities into the low-pressure system;
forming a differential value between the first pressure signal and the second pressure signal;
carrying out a comparison between the difference value and a difference threshold value;
determining a result of checking the functionality of the rebreather based on the comparison; and
providing an output signal which indicates the result of the check.

2. A process according to claim 1, wherein a first low pressure comparison of the measured values of the low pressure sensor at the first time, with a first lower low pressure threshold value, is carried out.

3. A process according to claim 1, wherein a second low pressure comparison of the measured values of the low pressure sensor at the second time, with a second lower low pressure threshold value, is carried out.

4. A process according to claim 1, wherein a third low pressure comparison of the measured values of the low pressure sensor at a third time, with a third lower low pressure threshold value, is carried out.

5. A process according to claim 1, wherein a continuous, metrological acquisition and recording of pressure measurement signals of a high pressure sensor arranged in or on a high pressure system takes place.

6. A process according to claim 5, wherein a high pressure comparison of measured values of the high pressure sensor at the first time, with a lower high pressure threshold value, is carried out.

7. A process according to claim 6, wherein the high-pressure comparison is performed to determine a pressure level in the high-pressure system or to determine a fluid tightness in the high-pressure system.

8. A process according to claim 1, wherein a computer program with non-transitory computer-readable media or computer program product with non-transitory computer-readable media for carrying out one or more of the process steps.

9. A process according to claim 1, wherein a control unit, comprising a processor and memory, is provided in or on the rebreather to carry out one or more of the process steps.

10. A device comprising:
means for continuously metrologically detecting and recording of pressure measurement signals of a low-pressure sensor arranged in or on a low-pressure system of the rebreather;
means for identifying, based on the pressure measurement signals of the low pressure sensor as to whether there is currently a situation in which an inflow of quantities of gas into the low pressure system has started;
means for storing a first pressure measurement signal at a first time, which indicates the identified pressure situation immediately after the start of the inflow of gas quantities into the low-pressure system with a breathing bag;
means for storing a second pressure measurement signal at a second time, which indicates a pressure situation at the end of the inflow of gas quantities into the low-pressure system;
means for forming a differential value between the first pressure signal and the second pressure signal;
means for carrying out a comparison between the difference value and a difference threshold value;
means for determining a result of checking the functionality of the rebreather based on the comparison; and
means for providing an output signal which indicates the result of the check.

11. A device according to claim 10, wherein the device is configured as a closed-circuit breathing apparatus, rebreather apparatus or rebreather diving apparatus, which comprises:
a control unit configured to provide one or more of said means for continuously metrologically detecting and recording, means for identifying, means for storing a first pressure measurement signal at a first time, means for storing a second pressure measurement signal at a second time, means for forming a differential value, means for carrying out a comparison, means for determining a result of checking, and means for providing an output signal;
a low-pressure system comprising a breathing bag, the low-pressure sensor, a valve arrangement, a hose line system, a breathing circuit, a mouthpiece, and a CO₂ absorber; and
a high-pressure system comprising a compressed gas supply, a pressure reducer, and the high-pressure sensor.

12. A device according to claim 11, wherein the control unit is configured to provide a first low pressure comparison of the measured values of the low pressure sensor at the first time with a first lower low pressure threshold value.

13. A device according to claim 11, wherein the control unit is configured to provide a second low pressure comparison of the measured values of the low pressure sensor at the second time, with a second lower low pressure threshold value.

14. A device according to claim 11, wherein the control unit is configured to provide a third low pressure comparison of the measured values of the low pressure sensor at a third time, with a third lower low pressure threshold value.

15. A device according to claim 11, wherein the control unit is configured to provide a continuous, metrological acquisition and recording of pressure measurement signals of a high pressure sensor arranged in or on a high pressure system.

16. A device according to claim 15, wherein the control unit is configured to provide a high pressure comparison of measured values of the high pressure sensor at the first time, with a lower high pressure threshold value.

17. A device according to claim 15, wherein the control unit is configured to perform the high-pressure comparison to determine a pressure level in the high-pressure system or to determine a fluid tightness in the high-pressure system.

18. A device according to claim 10, wherein a computer program with non-transitory computer-readable media or computer program product with non-transitory computer-readable media is executable with the control unit for one or more of:
continuously metrologically detecting and recording of pressure measurement signals of a low-pressure sensor arranged in or on a low-pressure system of the rebreather;
identifying, based on the pressure measurement signals of the low pressure sensor as to whether there is currently a situation in which an inflow of quantities of gas into the low pressure system has started;
storing a first pressure measurement signal at a first time, which indicates the identified pressure situation immediately after the start of the inflow of gas quantities into the low-pressure system with a breathing bag;
storing a second pressure measurement signal at a second time, which indicates a pressure situation at the end of the inflow of gas quantities into the low-pressure system;
forming a differential value between the first pressure signal and the second pressure signal;
carrying out a comparison between the difference value and a difference threshold value;
determining a result of checking the functionality of the rebreather based on the comparison;
providing an output signal which indicates the result of the check.

19. A rebreather device comprising:
a low-pressure system comprising a breathing bag, a low-pressure sensor, a valve arrangement, a hose line system, a breathing circuit, a mouthpiece, and a CO₂ absorber;
a high-pressure system comprising a compressed gas supply, and a pressure reducer; and
a control unit configured to:
acquire and record pressure measurement signals of the low-pressure sensor arranged in or on the low-pressure system of the rebreather;
identify, based on the pressure measurement signals of the low pressure sensor, whether there is currently a situation in which an inflow of quantities of gas into the low pressure system has started;
store a first pressure measurement signal at a first time, which first pressure measurement signal indicates the identified pressure situation immediately after the start of the inflow of gas quantities into the low-pressure system with a breathing bag;
store a second pressure measurement signal at a second time, which second pressure measurement signal indicates a pressure situation at the end of the inflow of gas quantities into the low-pressure system;
form a differential value between the first pressure signal and the second pressure signal;
carry out a comparison between the difference value and a difference threshold value;
determine a result of checking functionality of the rebreather based on the comparison; and
provide an output signal which indicates the result of the check.

20. A rebreather device according to claim 19, wherein the high-pressure system further comprises a high-pressure sensor arranged in or on a high pressure system and the control unit is further configured to:
acquire and record pressure measurement signals of the high pressure sensor; and
provide a high pressure comparison of measured values of the high pressure sensor at the first time, with a lower high pressure threshold value, wherein the high-pressure comparison is used to determine a pressure level in the high-pressure system or to determine a fluid tightness in the high-pressure system.
